(19) United States
(12) Patent Application Publication
(10) Pub. No.: US 2008/0171032 A1
(43) Pub. Date: Jul. 17, 2008

(54) DIETARY SUPPLEMENT COMPOSITION AND METHOD OF USE FOR THE TREATMENT AND PREVENTION OF OXIDATIVE STRESS

(76) Inventor: Gregory J. Kelly, Glastonbury, CT (US)

Correspondence Address: MCCARTER & ENGLISH LLP;CITYPLACE I, 185 ASYLUM STREET, HARTFORD, CT, 06103, US

(21) Appl. No.: 12/013,612

(22) Filed: Jan. 14, 2008

Related U.S. Application Data
(60) Provisional application No. 60/880,139, filed on Jan. 12, 2007.

(51) Int. Cl. A61K 38/44; A61P 3/02

(52) U.S. Cl. 424/94.4

(57) ABSTRACT

Described are compositions comprising a combination of superoxide dismutase and at least one member selected from the group consisting of astaxanthin, tocotrienol, carnosine, and benfotiamine, and methods of using the same for ameliorating or preventing the detrimental effects of oxidative stress.

### CROSS REFERENCE TO RELATED APPLICATIONS

Under 35 U.S.C. § 119(e) this application claims the benefit of U.S. Provisional Application No. 60/880,139 filed Jan. 12, 2007, and is hereby incorporated by reference in its entirety.

### FIELD OF THE INVENTION

This invention relates to dietary supplement compositions and methods of use thereof for the treatment and/or prevention of the detrimental effects of reactive oxygen species.

### BACKGROUND

The production of reactive oxygen intermediates (e.g., oxygen ions, superoxide, hydrogen peroxide, peroxynitrite, hydroxyl radicals, and nitric oxide) has been associated with profound damage to cellular components, for example, they oxidize and crosslink proteins, mutagenize DNA, and peroxidize lipids. In addition, once formed, free radicals can interact to produce other free radicals and non-radical oxidants such as singlet oxygen and peroxides. Degradation of some of the products of free radical reactions can also generate potentially damaging chemical species. For example, malondialdehyde is a reaction product of peroxidized lipids that reacts with virtually any amine-containing molecule.

These damaging effects can lead to pathological conditions such as, for example, skin, tissue, and organ disorders, such as atherosclerosis, arthritis, cytotoxicity, skin inflammation, photoaging, wrinkling, actinic keratosis, tumor formation, cancer; hypertension, lung disease, myocardial infarction; immune diseases, lupus, and scleroderma; neurodegenerative diseases such as Alzheimer's Disease and Parkinson's Disease; acquired immune deficiency syndrome (AIDS); cataracts; ischemic injury, and heart disease. In fact, free radicals have been implicated in over 50 diseases, and it has been estimated that at least 85% of chronic and degenerative diseases result from oxidative damage caused by free radicals.

The human body has several mechanisms to counteract damage by free radicals and other reactive oxygen species, including enzymes that catalytically scavenge the intermediates of oxygen reduction (e.g., superoxide dismutase, catalase, and peroxidase), and chemical antioxidants, which act as radical scavengers and convert the radicals to less reactive species. Many types of antioxidants are not generated endogenously and must be ingested or absorbed after external application.

Therefore, it is desirable to provide a composition that is able to protect from the damaging effects of the variety of reactive oxygen species that is, chemically stable over time, demonstrates high efficacy and bioavailability, and which is substantially free of detrimental side effects.

### SUMMARY

In one aspect the invention relates to a dietary supplement composition comprising superoxide dismutase and at least one member selected from the group consisting of astaxanthin, tocotrienol, carnosine, and benfotiamine. The particular combination of ingredients operates synergistically to inhibit the damaging effects of free radicals.

In certain aspects, the composition additionally comprises one or more pharmaceutically acceptable carriers, excipients, adjuvants, surfactants, emulsifiers, and the like. In another aspect, the composition according to the invention is particularly suitable for the oral administration to an individual.

In another aspect, the invention relates to methods of administering the composition of the invention to an individual for treating and/or preventing the deleterious effects of free radicals.

Additional objects and advantages of the present invention will be appreciated by one of ordinary skill in the art in light of the current description and examples of the preferred embodiments, and are expressly included within the scope of the present invention.

### DETAILED DESCRIPTION

In order for a composition to be effective in the elimination of a variety of free radicals, it must contain antioxidants that are specific for such free radicals. The present invention relates to the unexpected discovery that the combination of superoxide dismutase (SOD) and at least one of astaxanthin, tocotrienol and/or tocopherol, carnosine, benfotiamine operates synergistically to reduce of the harmful effects of free radicals. In addition, administration of the combination results in a surprising and unexpected improvement in skin health, and appearance. While not being limited by any particular theory, the inventors hypothesize that this effect is mediated by inhibiting the detrimental effects of free radicals.

In one aspect the present invention comprises a dietary supplement composition comprising a combination of superoxide dismutase (SOD) and at least one of astzanthin, tocotrienol and/or tocopherol, carnosine, benfotiamine, or a combination thereof. In certain embodiments, the composition comprises in a daily dose of SOD of from about 50 IU to about 1000 IU. In other embodiments, the composition comprises a daily dose of SOD of from about 100 IU to about 500 IU. In any embodiment the composition may comprise in a daily dose at least one of astaxanthin at from about 100 mg to about 400 mg; tocotrienols and/or tocopherols at from about 1 mg to about 100 mg; carnosine at from about 0.5 mg to about 10 mg; benfotiamine at from about 0.5 mg to about 10 mg; and para-aminobenzioc acid at from about 50 mg to about 500 mg or a combination thereof.

In an example of one embodiment, the composition comprises a daily dose of about 100 IU of SOD; about 200 mg of astaxanthin; about 25 mg total of tocopherol and tocotrienol; about 4 mg of carnosine; and about 4 mg of benfotiamine, and about 200 mg of para-aminobenzoic acid (Table I).

In certain embodiments, the composition also comprises at least one of a pharmaceutically acceptable carrier, excipients, adjuvant, a nutrient component; for example, a mineral, a vitamin, a lipid, an oil, an alcohol, a carbohydrate, a protein, for example, gliadin, albumin, or an enzyme; an amino acid, or a nucleic acid; a flavoring agent; a preservative; a coloring agent; a surfactant or emulsifier; or a combination thereof.

TABLE I
Examplary Formulations of the Dietary Supplement Composition.

| | | | Particularly |
|---|---|---|---|
| | | | Preferred |
| | Dose per | Daily | Embodiment |
| Ingredient | Capsule | Dosage | (Daily Dosage) |
| SOD | 25-500 mg | 50-1000 mg | 100 mg |
| (available as Glisodin ®) | | | |
| Astaxanthin | 50-200 mg | 100-400 mg | 200 mg |
| (available as | | | |
| AstraREAL ® Fuji | | | |
| Health Science, Inc.) | | | |
| Tocotrienol and | 1-25 mg | 2-50 mg | 25 mg |
| Tocopherol | | | |
| (available as Tocotab ™ | | | |
| P30, Fuji Health | | | |
| Science, Inc.) | | | |
| Carnosine | 0.5-10 mg | 1-20 mg | 4 mg |
| Benfotiamine | 0.5-10 mg | 1-20 mg | 4 mg |
| PABA | 50-250 mg | 100-500 mg | 200 mg |
| (para-aminobenzoic | | | |
| acid) | | | |

In another aspect, the invention comprises a method for treating or preventing a disease or condition attributed to oxidative stress and/or reactive oxygen species including, for example, skin, tissue, and organ disorders, such as atherosclerosis, arthritis, cytotoxicity, skin inflammation, photoaging, wrinkling, burns, sun burns, tissue wounds, tissue damage, sickle cell anemia, actinic keratosis, tumor formation, cancer; hypertension, lung disease, myocardial infarction; immune diseases, lupus, and scleroderma; neurodegenerative diseases such as Alzheimer's Disease and Parkinson's Disease; acquired immune deficiency syndrome (AIDS); cataracts; ischemic injury, and heart disease. In certain embodiments, the method comprises the step of administering an effective amount of the composition of the invention in one or more unitary doses in a pharmaceutically acceptable form to an individual in need thereof.

The invention comprises a method for improving skin health comprising administering to an individual an effective amount of a dietary supplement composition comprising superoxide dismutase and at least one of astaxanthin, tocotrienols and/or tocopherols, carnosine, benfotiamine, or a combination thereof, wherein the composition is divided into one or more dosage units.

Astaxanthin is a naturally occurring carotenoid pigment, and a powerful biological antioxidant. Astaxanthin exhibits strong free radical scavenging activity and protects cell membranes, cells, and tissues against lipid peroxidation and oxidative damage. Astaxanthin has been shown to protection against oxygen-induced mitochondrial membrane damage. The administration of astaxanthin at 1 mg/100 g of body weight is believed to inhibit lipid peroxidation, thereby protecting mitochondrial membranes. Damage to mitochondrial DNA is often implicated in the aging process, since damaged mitochondrial cells are unable to produce energy needed for cells to sustain proper function.

Tocotrienols and tocopherols are powerful antioxidants that, together, compose the vitamin E family. Studies have shown that tocotrienols protect cells from oxidative damage to a greater extent than other members of the vitamin E family. A randomized, placebo-controlled human study investigating the antioxidant effects of tocotrienols demonstrates that tocotrienol supplementation (at 40 mg/day) is more effective in lowering serum lipid oxidation products than placebo. Oxidation of membrane lipids in skin cells is associated with skin aging. The inventors hypothesize that the protective effects of tocotrienols against oxidation are likely to help protect skin cells, and improve the skin's condition and appearance. While tocopherols are generally present in common vegetable oils (i.e. soy, canola), tocotrienols, on the other hand, are concentrated in cereal grains (ie. oat, barley, and rye, rice bran), with the highest level found in crude palm oil. Commercial tocotrienols (d- and d,l-tocotrienols) and tocopherols are mainly obtained from natural sources, such as palm or rice bran oil or synthesized.

Camosine is recognized as an antioxidant, a membrane-protective compound, and a free radical scavenger. In addition, carnosine has been shown to inhibit the formation of cross-links in proteins (specifically collagen), which is associated with aging. Furthermore, carnosine promotes collagen deposition and maintains cellular integrity by affecting protein metabolism. This is important to skin because collagen is a key component of skin and is responsible for its healthy appearance and elasticity. A study in human fibroblasts (connective tissue that secretes collagen) demonstrates that cells exposed to 50 mM carnosine experience a reduction in onset of cell senescence. Conversely, when cells approaching senescence are supplemented with carnosine, they are markedly rejuvenated and their lifespan extended.

Superoxide dismutases (SODs) have been studied in the treatment of numerous diseases since their characterization in 1968 by McCord and Fridovich (J. Biol. Chem., 1969, 244, 6049-6055). SOD is, in fact, an enzyme which promotes removal of the superoxide radical (O₂⁻) by dismutation and therefore constitutes a system for providing protection from the deleterious effects of this radical, which is capable of forming in vivo from atmospheric oxygen. Consequently this enzyme plays a fundamental role in preventing the toxic effects which could result from exposure of the cells and the organism to an oxygenated atmosphere in which oxygen (a biradical) loses an unpaired electron (reduction).

As free radicals are involved in numerous diseases, the use of SOD in therapeutics has been recommended in different inflammatory processes (rheumatism and fibrosis in particular), viral processes (HIV infection in particular) and toxic conditions associated with the presence of substantial amounts of oxygen (central nervous system, ischemia, non-vascular gastrointestinal disorders, eye disorders or control of the undesirable effects of anticancer treatments) (Greenwald R. A., Free Radical Biol. Med., 1990, 8, 201-209). The free forms of SOD which have been tested are Cu,Zn-SOD (vegetable origin or animal origin: bovine, rat or human), Mn-SOD (human, vegetable, algal origin), Fe-SOD, and recombinant SOD. The plasmatic half-lives of native SODs are very variable (of the order of a few minutes for Cu,Zn-SOD; of the order of several hours for Mn-SOD, for example).

Different modified forms for parenteral administration have been proposed for increasing the plasmatic half-life of these SODs; modified forms which may be mentioned are SODs conjugated with polyethylene glycol (SOD-PEG), SODs conjugated with heparin (SOD-heparin), SODs conjugated with albumin (SOD-albumin), SOD polymers or copolymers and liposomal SODs. However, these different SODs have the major disadvantage of being very poorly absorbed when administered orally.

The compositions of the instant claims contemplate administration of an orally available SAD. An effective orally bioavailable superoxide dismutase is currently commercially available as Glisodin®. SOD is typically inactivated in the digestive tract in its free form. However, Glisodin®'s antioxidant activity is able to be preserved due to coupling with gliadin, a glycoprotein, present in wheat and some other cereals. A double-blind, randomized, placebo-controlled human study demonstrated that the gliadin-SOD mixture (at 1000 IU/day) protects against DNA damage. Damage to nuclear and mitochondrial DNA is one of the prime causes of cellular disfunction. In addition, GliSODin® was shown to reduce skin redness following UV-irradiation in a human, placebo-controlled study. It is therefore believed to protect skin cells from solar damage. In addition, animal studies have demonstrated GliSODin®'s ability to elevate the activity of antioxidant enzymes and to increase cellular resistance to oxidative stress-induced damage at 90 IU/mg of body weight. Oxidative damage of proteins, lipids, and DNA may be related to skin erythema, wrinkles, photoaging, and loss of elasticity.

Benfotiamine is believed to have anti-aging effects due to its ability to protect cells from harmful metabolic end-products known as advanced glycation end-products (AGEs). Benfotiamine is a lipid-soluble form of thiamine which is able to supple very high intracellular levels of thamine upon administration, while supplementation with thiamine is ineffective because it is poorly absorbed and metabolized by cells. The thiamine delivered by benfotiamine is then able to inhibit the formation of AGEs, which are involved in protein degradation and the resulting signs of aging. Administration of benfotiamine at 600 mg/day prior to blood draw results in a significant decrease of both glycoxidation and AGE formation in the cells of test subjects. AGE in cells contributes to the formation of free radicals, resulting in oxidative damage. Furthermore, the long-lived protein collagen is especially vulnerable to AGE formation and damage due to its slow turnover rate. In compositions of the invention, benfotiamine's ability to protect against AGE formation appears to be significant for skin structure and health.

Para-aminobenzoic acid (PABA) is a non-protein amino acid that is widely distributed in nature. It is sometimes referred to as vitamin Bx, but it is neither a vitamin nor an essential nutrient for humans. PABA is an intermediate in the synthesis of folic acid in bacteria. The sulfonamide antibiotics are structurally similar to PABA and interfere with the synthesis of nucleic acids in sensitive micro-organisms by blocking the conversion of PABA to the co-enzyme dihydrofolic acid, a reduced form of folic acid. In humans, dihydrofolic acid is obtained from dietary folic acid; thus sulfonamides do not affect human cells. PABA is also known as 4-aminobenzoic acid. It is a solid substance with slight solubility in water. PABA may have antifibrosis activity.

PABA and para-aminobenzoate potassium are available from numerous manufacturers generically. Branded products for para-aminobenzoate potassium include M2 Potassium (Miller Pharmacal) and Potaba (Glenwood). Following ingestion, PABA is passively absorbed mainly from the small intestine. From there, it enters the portal circulation. Some metabolism of PABA occurs in the liver. A major metabolite is N-acetyl PABA. Pharmaceutical doses of PABA are indicated for Peyronie's disease, scleroderma, morphea and linear scleroderma. There is less evidence to indicate it for pemphigus and dermatomyositis.

A retrospective review analyzed skin responses of scleroderma patients to potassium para-aminobenzoate therapy. Ninety percent of 224 patients treated with about 12 grams daily of potassium para-benzoate (POTABA) experienced mild, moderate or marked skin softening. Among a parallel group of 96 evaluable scleroderma patients who did not receive potassium para-benzoate, less than 20% had mild or moderate improvement at the end of follow-up. The difference in skin softening in the treated group, compared to the untreated group, was statistically significant, and no significant adverse events were reported. Again, these findings require confirmation. Clinical improvement was noted in two dermatomyositis patients treated with 15 to 20 grams of potassium para-benzoate daily. Adequate clinical trials are necessary before any conclusion can be drawn regarding the possible effectiveness of PABA for dermatomyositis.

In any of the embodiments disclosed herein, the dietary supplement may include at least one additional nutrient component. A nutrient may include, for example, minerals, for example, calcium, magnesium, chromium, copper, iodine, iron, manganese molybdenum, selenium, zinc, boron, sodium, potassium, silicon; carotenoids, beta-carotene, grape seed extract, oligomeric proanthocyanidins (OPCs) hyaturonic acid, L-cysteine, Allantoin, glycolic acid, collagen elastin antibiotic peptides, ascorbyl palmitate, aloe vera extract, magnesium ascorbyl phosphate, ascorbyl acetate, lutein, zeaxanthin, lycopene, choline, linoliec acid, linolenic acid, para-aminobenzoic acid, alpha-lipoic acid, flavonoids, coenzyme Q10, indolinic or quinolinic aminoxyls, catechins, essential oils; oils, for example, lipids, phospholipids, triglycerides, inositols; salts, methylsulfonyl methane, spirulina; vitamins, for example, A, B and B-complex vitamins, C, D, E, K, thiamine, riboflavin, niacin, pantothenic acid, pyridoxine, folic acid, biotin, derivatives thereof, amino acids, for example, arginine, histidine, lysine, isoleucine, leucine, methionine, phenylalanine, threonine, tryptophan, tyrosine, valine, aspartate, glutamate, serine, proline, asparagine, glutamine, cysteine, glycine, and alanine; and the like.

Grape seed extract contains oligomeric proanthocyanidins (OPCs) which are among the most powerful antioxidants at preventing free radical damage. It helps our body resist tissue deterioration, inflammation and other oxidative damage. In addition, Grape Seed Extract helps preserve collagen and elastin integrity in the skin by inhibiting collagenase and elastinase (enzymatic breakdown).

Hyaluronic Acid plays an important role in tissue hydration, lubrication and cellular function, and is able to hold more water than any other natural substance. Its unmatched hydrating properties result in increased smoothness, softening and decreased wrinkles.

L-Cysteine & L-Cystine are critical precursors to collagen and essential for maintaining skin elasticity.

Alpha Lipoic Acid is both lipid and water soluble and works on every part of the cells. It helps eliminate damaged collagen resulting in erasing wrinkles and facial scars. It also helps prevent pre-mature aging and skin damage by inhibiting sugar from binding to protein.

Aloe Vera contains amino acids and assorted alkaloids that help soothe and moisturize the skin while balancing skin pH. Allantoin is an antioxidant that helps fight free-radicals, the scavengers that damage skin and speed the aging process. Glycotic Acid, makes skin peeling or exfoliation possible. Eliminating dead cells on the surface revealing new skin underneath.

Vitamins A, C & E protect, enrich, and soften the skin with an anti-inflammatory effect.

Natural Antibiotic Peptides are effective against different types of bacteria commonly found in human skin infections and acne such as Escherichia Coli, Staphylococcus Aureus, Pseudomanas Aeruginosa and Acne vulgaris. These natural antibiotics will not cause any bacterial resistance as pharmaceutical antibiotics do.

In any of the embodiments disclosed herein, the dietary supplement of the invention may include a flavoring agent. A flavoring agent may include, for example, anethole, anise oil, benzaldehyde, blackberry, blueberry, caraway, caraway oil, cardamom oil, cardamom seed, cherry juice, cherry syrup, cinnamon, cinnamon oil, an alcohol, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, and wild cherry syrup.

In any embodiment of the composition of the invention described herein, the composition may include a suitable means for administering the composition to a person. Potential means of administering the composition include, for example, a gel, lotion, shampoo, spray, powder, pill, tablet, emulsion, liquid, salts, pastes, jellies, aerosols, ointments, capsules, gel caps, controlled release capsules or any other suitable form that will be obvious to one of ordinary skill in the art.

In other embodiments of the composition of the present invention includes the use of one or more inert ingredients such as a pharmaceutically acceptable excipients, and/or carriers to create a suitable form or consistency for administration to patients. Examples of inert ingredients that may be used in any of the embodiments of the composition of the invention include, dicalcium phosphate, stearic acid, microcrystalline cellulose, croscarmellose sodium, magnesium stearate, silicon dioxide, maltodextrin, dextrin, olive oil, corn starch, gum acacia, sodium carboxymethylcellulose, medium-chain triglycerides, vitamin C palmitate, polyglycerol fatty acid esters, calcium silicate, soy lecithin, rosemary extract, sodium caseinate, titanium dioxide, hydroxypropyl methyl cellulose, gelatin, glycerin, soybean oil, beeswax, lecithin, corn oil and the like.

In another aspect the composition of the invention may optionally contain one or more preservatives to prolong product shelf-life. As one of ordinary skill in the art will recognize any of the above-described inert ingredients may or may not be added in any suitable combination without limiting the scope of the present invention.

The following is a discussion of examples of physiological mechanisms related to skin health for which the composition of the invention is useful.

Collagenase and gelatinase are metalloenzymes found in their latent forms. Hypochlorous acid, generated in any inflammatory reaction, reacts with the latent forms of these two enzymes to convert them to an active form. Collagenase and gelatinase are believed to facilitate the migration of inflammatory cells into the area of the inflammatory focus. It is postulated that the enhancement of antioxidants specific for the amelioration of hydrogen peroxide and hypochlorous would reduce the concentrations of collagenase and gelatinase which are activated.

Burn wounds to skin and other organs can occur by ultraviolet radiation (uv), chemical agents, conductive or convective heat, electrocution, etc. Burns can occur in lung parenchyma by the inhalation of smoke or caustic gases (see section on tissue injury). Burn wounds to the skin are graded as first, second, and third degree burns (the most severe). It is postulated that any burn wound produces tissue damage, largely by the production of oxidants. Liposomes (artificial membranes) when exposed to uv undergo peroxidation. It has been postulated that similar peroxidation occurs in skin when it is exposed to uv radiation. Exposure of skin to uv varies in intensity and length of exposure. Daily exposure to uv (e.g., sunlight) has been postulated to result in skin wrinkling. Depending on the intensity and/or length of skin exposure to uv light, first, second or third degree burns can result.

Hairless mice exposed to a single exposure of uv resulted in a broad range decrease of antioxidants: glutathione, beta-carotene, alpha-tocopherol. The enzyme activity of catalase and glutathione reductase were also decreased. These decreases in the concentration of antioxidants and enzyme activity in skin due to uv exposure supports the concept of the occurrence of free radicals in skin. It is postulated that lipid peroxidation could be inhibited by an enhancement of antioxidants in skin.

In experimentally produced severe burns, remote organ injury is observed (e.g. microvascular injury). It is postulated that it is the activation of leukocytes, the production of cytokines, and the pathological production of prostaglandins which are responsible for the damage seen in burn wounds. Excessive free radical production has been cited as a factor in delayed wound healing. It is postulated that topical, aerosol, or intravenous administration of antioxidants would ameliorate the effects of pathologic oxidants and prostaglandin production as well as promote wound healing in various skin injuries.

Tissue injury occurs as a result of an inflammatory focus occurring in the area of a cell or an organ. Inflammation can occur due to a local inducement (e.g. hepatitis) or due to an injury occurring to one organ in a remote location and another discontiguous organ which also sustains an injury (e.g., severe burns occurring to skin (the first organ) with subsequent injury to the lungs (the second organ)). In either case, local or remote tissue injury is believed to be mediated by activated leukocytes which release oxidants. Oxidants released from leukocytes react with cellular (organ) membranes. Repeated cellular membrane exposure to oxidants decreases antioxidant levels, which increases their susceptibility to damage. Increasing the levels of antioxidants in the extracellular and/or intracellular, and/or the lipid-aqueous interface is postulated to thwart oxidant damage to vital cellular structures.

Inflammation can arise from infective agents (e.g. virion), trauma, chemical agents, immune reactions, metallic agents, ionizing or thermal agents. The sine qua non of inflammation are heat, redness, edema pain and loss of function (e.g., of the surrounding tissue). In any type of inflammation, characteristic inflammatory cells can be found, for example leukocytes, eosinophils, macrophages/macrocytes. Each of these cell types produce free radicals as part of a programmed response. Also as part of that “programmed” response are the production of cytokines, such as TNF-•, CM-CSF and IL-6. These particular cytokines promote the production of oxidants. Oxidants are also generated as a byproduct of prostaglandin production, which is part of the propagation and amplification of the inflammatory process. Platelets are also involved in the inflammatory process by virtue of their ability to act as a plug (as in a clot); but also due to their liberation of platelet activating factor (PAF), which liberates arachidonic acid (i.e., the major precursor of pro-inflammatory lipids) from leukocytes.

The production of prostaglandins is dependent upon the free radical tone (or concentration) of the microenvironment and metabolite synthesis. By decreasing the free radical tone and PG free radical intermediate metabolites, it is postulated that the pathological production of prostaglandins would be reduced, the amplification effect that PGs have as a role in the inflammatory process could be limited. Theoretically, either the lipooxygenase limb or the cyclooxygenase limb of the prostaglandin pathway could be effected by an increased ratio of water soluble antioxidants to fat soluble antioxidants, or fat soluble antioxidants to water soluble antioxidants.

Free radicals or oxidants also have a plethora of different effects on the tissue in which it occurs, e.g. membrane damage, platelet adhesion, blood vessel intimal damage, etc. By artificially increasing the antioxidant levels in areas where inflammation is occurring, it is postulated that the propagatory effect, tissue damage and pathologic physiologic reactions would be curtailed as well. The NF-KB transcription protein regulates the expression of a number of genes for proteins and cytokines involved in the inflammatory process. The activity and affinity that the NF-KB protein has for DNA is also regulated by GSH level. Enhancing levels of GSH decreases the activity and binding of NF-KB to DNA. It is postulated that by enhancing GSH levels that those cytokines and proteins involved in the inflammatory process would be decreased.

Ischemia, which is low tissue oxygen saturation of a given tissue, can occur in any organ system. All organs require a blood supply in order to remain viable. The intact organ whose arterial supply is compromised (either by partial or total occlusion) is rendered ischemic (e.g., coronary artery occlusion, organs awaiting transplantation, cerebral vascular accident, compartment syndrome, etc.). There are reversible and irreversible histological, physiological and biochemical changes which occur as a result of ischemic injury to tissue. End stage ischemia is universal and demonstrates necrosis. It has been theorized that the necrosis observed in ischemic tissue was due to oxidants generated by the uncoupling of the oxidative phosphorylation chain in mitochondria.

Direct evidence of free radical production as a result of ischemia was provided by Zweir et al. (Proc. Natl. Acad. Sci. USA, vol. 84, pp: 1404-1407) by the use of electron spin resonance spectroscopy. In reperfusion studies Zweir was able to show the alteration of one of the free radicals with the use of superoxide dismutase (which eliminated superoxide). In ischemic cardiac myocyte a depletion of ATP induces the release of arachidonic acid and palmitic acid. It is postulated that enhancement of tissue antioxidants would eliminate the superoxide free radical, as well as other oxidants that are not double produced as a result of ischemia and prostaglandin metabolite production; vitamin E would tend to inhibit the activity of phospholipase A2, whereas niacin would tend to inhibit the function of phospholipase C.

Sickle cell anemia is a genetically determined disease. Analysis of sickle cell patients RBC (HbS) demonstrates a number of peculiarities of the membrane: frozen spectrin shell of irreversibly sickled RBC, an abnormal orientation of the lipid bilayer phospholipids, deficient calcium-ATPase, a propensity for HbS RBCs to adhere to vascular endothelium, and oxidized thiol groups on the HbS molecule. It is the characteristic of the tendency of adherence to the vascular endothelium which is the likely primary pathogenesis of the disease, which is vasooclusion of the microvasculature. Consequently, ischemic injury occurs to organs. Additional evidence of free radical damage to HbS are a deficiency of alpha-tocopherol, increased amounts of malondialdehyde, and abnormal group cross linking by malonadehyde. Superoxide anion can enter into erthrocytes via anion channels, resulting in the formation of methemoglobin and the ultimate lysis of erythrocytes. Sickle RBCs spontaneously generate sixty percent greater quantities of superoxide and approximately 75% more hydrogen peroxide when compared with controls. Superoxide dismutase is increased by about 50%, glutathione peroxidase and catalase were decreased by approximately 50 and 29% respectively. Glutathione and vitamin E levels were significantly reduced. It is postulated that by increasing both bone narrow and serum antioxidant levels that free radicals produced by sickled RBCs would be markedly reduced. Vitamin E levels have been found to be difficult to augment by oral administration. Increasing antioxidant levels in RBCs and in plasma by intravenous administration at the time of a crisis (an ischemic event), it is postulated that RBCs would be less sticky and less prone to adhere to the microvasculature intimal lining. Given that plasma levels of antioxidants were to remain high, it would be postulated that there would be less damage due to ischemia (e.g., decrease the extent of a cerebral vascular accident or decrease the extent of pain which is due to ischemia, etc.).

Additional objects and advantages of the present invention will be appreciated by one of ordinary skill in the art in light of the current description and examples of the preferred embodiments, and are expressly included within the scope of the present invention.

It is understood that the examples and embodiments described herein are for illustrative purposes only and that various modifications or changes in light thereof will be suggested to persons skilled in the art and are included within the spirit and purview of this application and are considered within the scope of the appended claims.

We claim:

1. A dietary supplement composition comprising a combination of superoxide dismutase, astaxanthin, tocotrienol, tocopherol, carnosine, benfotiamine, and para-aminobenzioc acid.

2. The dietary supplement composition of claim 1, wherein the supplement comprises on a daily dosage basis from about 50 IU to about 1000 IU of superoxide dismutase.

3. The dietary supplement composition of claim 1, wherein the supplement comprises on a daily dosage basis from about 100 mg to about 400 mg of astaxanthin.

4. The dietary supplement composition of claim 1, wherein the supplement comprises on a daily dosage basis from about 1 mg to about 50 mg of tocotrienol.

5. The dietary supplement composition of claim 1, wherein the supplement comprises on a daily dosage basis from about 0.5 mg to about 20 mg of carnosine.

6. The dietary supplement composition of claim 1, wherein the supplement comprises on a daily dosage basis from about 0.5 mg to about 20 mg of benfotiamine.

7. The dietary supplement composition of claim 1, wherein the supplement comprises on a daily dosage basis from about 1 mg to about 50 mg of tocopherol.

8. The dietary supplement composition of claim 1, wherein the supplement comprises on a daily dosage basis from about 50 mg to about 400 mg of para-aminobenzoic acid.

9. The dietary supplement composition of claim 1, further comprising at least one of a pharmaceutically acceptable carrier or vehicle, exciptient, adjuvant, inert ingredient, a cellulose fiber, flavoring agent, coloring agent, protein, nutrient, binder, gum, polyol, alcohol, polymer, plasticizer, lipid, oil, surfactant, emulsifier, carbohydrate, stabilizer, sweetener, diluent, zolubilizing agent an additional antioxidant, herbal extract or combination thereof.

10. The dietary supplement of claim 2, wherein the additional antioxidant comprises at least one of a indolinic or a quinolinic aminoxyl, a flavonoid, a flavonol, a flavone, tocopherol, ascorbic acid, a catechin, a polyphenol, a phytosterol, quercetin, an essential oil, a quinone, an isoprenoid, a carotenoid, glutathione or combination thereof.

11. A method of preventing or treating a disease or an injury in an individual induced by pathological free radical reactions, the method comprising administering an effective amount of an antioxidant composition of claim 1, wherein the composition is administered in a pharmaceutically acceptable form.

12. The method of claim 10, wherein the disease or injury is selected from the group consisting of: skin inflammation, photoaging, wrinkles, burns, sun burns, tissue wounds, tissue damage, actinic keratosis, and scleroderma; and tissue injury induced by a chemical agent or a caustic agent, and wherein the antioxidant composition quenches free radicals and reduces the damage induced by the pathological free radical reactions.

13. The method of claim 10, wherein said composition is administered by a route of administration selected from the group consisting of: parenteral, enteral, oral, nasal, intravenous, intraperitoneal, subcutaneous, intramuscular, intraarticular, intraarterial, intracerebral, intracerebellar, intrabronchial, intrathecal, topical, and aerosol route.

14. The method of claim 10, wherein said pharmaceutically acceptable form is chosen from the group consisting of a gel, a lotion, a shampoo, a spray, a powder, a pill, a tablet, an emulsion, a liquid, a salt, a paste, a jelly, an aerosol, an ointment, a capsule, a gel cap, a control release capsule, a capsule containing an enteric coating, and any combination thereof.

15. A dietary supplement composition for the improvement of skin health comprising, on a daily dosage basis, from about 25 IU to about 200 IU of superoxide dismutase, from about 150 mg to about 250 mg of astaxanthin, from about 5 mg to about 50 mg of tocotrienol, from about 5 mg to about 50 mg of tocopherol, from about 1 mg to about 10 mg camosine, from about 1 mg to about 10 mg benfotiamine, and from about 100 mg to 250 mg of para-aminobenzoic acid.

16. The dietary supplement composition of claim 14, further comprising at least one of a pharmaceutically acceptable carrier or vehicle, excipient, adjuvant, inert ingredient, a cellulose fiber, flavoring agent, coloring agent, protein, nutrient, binder, gum, polyol, alcohol, polymer, plasticizer, lipid, oil, surfactant, emulsifier, carbohydrate, stabilizer, sweetener, diluent, zolubilizing agent an additional antioxidant, herbal extract or combination thereof.

17. A method for improving the health or appearance of the skin of an individual comprising the steps of administering, on a daily dosage basis, a composition comprising a combination of from about 75 IU to about 125 IU of superoxide dismutase, from about 175 mg to about 225 mg of astaxanthin, from about 10 mg to about 20 mg of tocotrienol, from about 10 mg to about 20 mg of tocopherol, from about 1 mg to about 5 mg carnosine, and from about 1 mg to about 5 mg benfotiamine, and from about 175 mg to 225 mg of para-aminobenzioc acid, wherein the composition is administered orally in a pharmaceutically acceptable form.

18. A dietary supplement composition comprising of a combination of an orally bioavailable superoxide dismutase composition, astaxanthin, tocopherol, tocotrienol, carnosine, benfotiamine, para-amino benzoic acid, grape seed extract, hyaluronic acid, L-cysteine, aloe vera extract, and alpha lipoic acid.

19. The dietary supplement composition of claim 20, wherein the composition comprises on a daily dosage basis about 200 mg of astaxanthin, about 100 mg of superoxide dismutase, about 25 mg total of tocopherol and tocotrienol, about 200 mg of para-aminobenzoic acid, about 4 mg of camosine, and about 4 mg of benfotiamine.
